(19) United States
(12) Patent Application Publication
(10) Pub. No.: US 2015/0086654 A1
(43) Pub. Date: Mar. 26, 2015

(54) NUTRITIONAL ENERGY AND FOCUS SUPPLEMENT

(71) Applicants: Hany Helmi, Statesboro, GA (US); Andrew Pandya, Savannah, GA (US)

(72) Inventors: Hany Helmi, Statesboro, GA (US); Andrew Pandya, Savannah, GA (US)

(21) Appl. No.: 14/493,188

(22) Filed: Sep. 22, 2014

Related U.S. Application Data
(60) Provisional application No. 61/880,408, filed on Sep. 20, 2013.

(51) Int. Cl. A61K 36/258; A61K 31/7068; A61K 31/714; A61K 31/197; A61K 31/519; A23L 1/29; A61K 31/198; A61K 31/194; A61K 31/455; A61K 31/19; A61K 31/4415; A61K 31/522; A61K 31/365

(52) U.S. Cl. CPC A61K 36/258; A61K 31/522; A61K 31/7068; A61K 31/714; A61K 31/197; A61K 31/519; A61K 31/365; A61K 31/198; A61K 31/194; A61K 31/455; A61K 31/19; A61K 31/4415; A23L 1/296; A23V 2002/00
USPC 424/728

(57) ABSTRACT

Embodiments of the present invention relate to a nutritional energy supplement for facilitating a healthy lifestyle and promoting mental focus. In one embodiment, a nutritional energy supplement has a set of active ingredients, the active ingredients comprising: between 40 mg to 480 mg of caffeine; between 62.5 mg to 900 mg of citicoline; between 0.000 mg to 0.03 mg of cyanocobalamin; between 3 mg to 36 mg ethylenediaminetetraacetic acid; between 0.1 mg to 1.2 mg of folic acid; between 10 mg to 120 mg of ginseng; between 131.25 mg to 1575 mg of glucuronolactone; between 93.75 mg to 1125 mg of L-Phenylalanine; between 100 mg to 1200 mg of malic acid; between 100 mg to 1500 mg of n-acetyl 1-tyrosine; between 8.75 mg to 105 mg of niacin; between 7.5 mg to 90 mg of potassium sorbate; and between 0.5 mg to 6 mg of pyridoxine.

### CROSS-REFERENCE TO RELATED APPLICATIONS

This application claims priority to U.S. Provisional Patent Application Ser. No. 61/880,408, filed Sep. 20, 2013, entitled “Nutritional Energy Supplement,” the disclosure of which is incorporated by reference herein in its entirety.

### BACKGROUND OF THE INVENTION

1. Field of the Invention

Embodiments of the present invention generally relate to a nutritional energy and focus supplement. More specifically, embodiments of the present invention relate to a nutritional energy and focus supplement having a balanced ratio of nutritional elements to facilitate a healthy lifestyle and promote mental focus.

2. Description of the Related Art

There are countless nutritional supplements, or nutraceuticals, in the marketplace; each one claiming to be better than all others. Most of these nutritional supplements are nothing more than a multivitamin tablet, providing the consuming individual a proper balance of daily recommended minerals and vitamins. A proper diet, and nothing more, could achieve the same results as many of the nutritional supplements on the market.

Certain nutritional supplements are more specialized, and thus, are generally not easily substituted with a traditional healthy diet. For example, nutritional supplements for significant muscle growth, erectile dysfunction, etc., often comprise combinations of ingredients that may not be readily available from ordinary foods. As such, these nutritional supplements often provide benefits to even the most healthy individuals.

Other nutritional supplements, which claim to provide certain benefits may not be healthy or beneficial in the slightest. For example, several “energy drinks” which hold themselves out as having no negative side effects, are often later found criticized in public for being the cause of headaches, pain or worse. In particular, some energy drinks have so much sugar and caffeine, that when consumed in just small doses, is more harmful than several cups of coffee. In addition to the caffeine, some possess intensely high levels of various nutrients and vitamins, e.g., at 2.000% the daily recommended value in one serving, that long-term side effects of regular consumption of the same may be toxic.

As such, there is a need for a nutritional energy and focus supplement having a balanced ratio of nutritional elements to facilitate a healthy lifestyle and promote mental focus.

### SUMMARY

Embodiments of the present invention generally relate to a nutritional energy and focus supplement. More specifically, embodiments of the present invention relate to a nutritional energy and focus supplement having a balanced ratio of nutritional elements to facilitate a healthy lifestyle and promote mental focus.

In one embodiment of the present invention, a nutritional energy and focus supplement has a set of active ingredients, the active ingredients comprising: between about 40 mg to about 480 mg of caffeine; between about 62.5 mg to about 900 mg of citicoline; between about 0.000 mg to about .03 mg of cyanocobalamin; between about 3 mg to about 36 mg ethylenediaminetetraacetic acid; between about 0.1 mg to about 1.2 mg of folic acid; between about 10 mg to about 120 mg of ginseng; between about 131.25 mg to about 1575 mg of glucuronolactone; between about 93.75 mg to about 1125 mg of L-Phenylalanine; between about 100 mg to about 1200 mg of malic acid; between about 100 mg to about 1500 mg of n-acetyl 1-tyrosine; between about 8.75 mg to about 105 mg of niacin; between about 7.5 mg to about 90 mg of potassium sorbate; and between about 0.5 mg to about 6 mg of pyridoxine.

In yet another embodiment, a nutritional energy and focus supplement has a set of inactive ingredients, the inactive ingredients comprising: between about 47.5 mg to about 570 mg of sucralose; between about 7.5 mg to about 90 mg of sodium benzoate; between about 10 mg to about 120 mg of sodium chloride; between about 131.25 mg to about 1575 mg of taurine; between about 0.02 ml to about 0.5 ml of flavorant; and between about 0.001 ml to about 180 ml of water.

### DETAILED DESCRIPTION

It is understood that the embodiments of the present invention are not limited to the particular methodologies, protocols, solvents and reagents, and the like, described herein as they may vary. It is also to be understood the terminology used herein is used for the purpose of describing particular embodiments only and not intended to limit the scope of the present invention. It must also be noted that as used herein, the singular form “a,” “an” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meaning as commonly understood by one of ordinary skilled in the art to which this invention belongs. Exemplary methods, devices, elements, ingredients and materials are described, although any methods and materials similar or equivalent to those described herein could be used in the practice or testing of the present invention. All references cited herein are incorporated by reference herein in their entirety.

In accordance with embodiments of the present invention, the nutritional energy and focus supplement (hereinafter “supplement”) comprises a set of active ingredients and a set of inactive ingredients. The active ingredients are those which provide a targeted health-related or energy-related function to the user upon consumption, and the inactive ingredients are those which complement the active ingredients with flavor, consistency, capsulation, or non-targeted health-related function.

In one embodiment of the present invention, the supplement may comprise a set of active ingredients, including caffeine, citicoline, cyanocobalamin, EDTA, folic acid, ginseng, glucuronolactone, L-Phenylalanine, malic acid, n-acetyl 1-tyrosine, niacin, potassium sorbate, pyridoxine, sucralose, sodium benzoate, sodium chloride, and/or taurine. In another embodiment, the supplement may comprise a set of inactive ingredients, including flavorants, sugars or sweeteners, and water.

In terms of commercially prepared volumes, a supplement may comprise between about 750 to about 10,000 mg of active ingredients. In one particular embodiment, the supplement comprises about 3,000 mg of active ingredients. For purposes of the disclosure herein, the percentages of ingredients shown are generally provided in light of active ingredients by volume as any number of inactive ingredients may be added without deviating from embodiments of the present invention.

Caffeine is a central nervous system stimulant, temporarily warding off drowsiness and restoring alertness in most individuals. While it is the world's most widely consumed psychoactive drug, unlike many other psychoactive substances, it is legal and unregulated in nearly all parts of the world. In North America, 90% of adults consume caffeine daily.

In many embodiments of the present invention, a supplement comprises between about 1.33% to about 15.92%, or between about 40 mg to about 480 mg of caffeine. In one particular embodiment, the supplement comprises about 5.31%, or about 160 mg of caffeine.

Citicoline, which is also known as cytidine diphosphate-choline (CDP-Choline) & cytidine 5′-diphosphocholine is a psychostimulant or nootropic. It is an intermediate in the generation of phosphatidylcholine from choline. Studies suggest that CDP-choline supplements increase dopamine receptor densities, and suggest that CDP-choline supplementation helps prevent memory impairment resulting from poor environmental conditions. Preliminary research has found that citicoline supplements help improve focus and mental energy and may possibly be useful in the treatment of attention deficit disorder. Citicoline has also been shown to elevate ACTH independently from CRH levels and to amplify the release of other HPA axis hormones such as LH, FSH, GH and TSH in response to hypothalamic releasing factors. These effects on HPA hormone levels may be beneficial for many individuals.

In many embodiments of the present invention, a supplement comprises between about 2.07% to about 24.88%, or between about 62.5 mg to about 900 mg of citicoline. In one particular embodiment, the supplement comprises about 16.58%, or about 500 mg of citicoline.

Cyanocobalamin is the most common and widely produced of the chemical compounds that have vitamin activity as vitamin B12, and may as such B12 may be utilized herein in lieu thereof. B12 is a water-soluble vitamin with a key role in the normal functioning of the brain and nervous system, and for the formation of blood. It is normally involved in the metabolism of every cell of the human body, especially affecting DNA synthesis and regulation, but also fatty acid synthesis and energy production.

In many embodiments of the present invention, a supplement comprises between about 0.00025 mg to about 0.03 mg, or generally negligible amount of cyanocobalamin. In one particular embodiment, the supplement comprises about 0.01 mg of cyanocobalamin.

EDTA, or ethylenediaminetetraacetic acid, is used to bind metal ions in the practice of chelation therapy, e.g., for treating mercury and lead poisoning, and is commonly used in a similar manner to remove excess iron from the body. In many embodiments of the present invention, a supplement comprises between about 0.10% to about 1.19%, or between about 3 mg to about 36 mg of EDTA. In one particular embodiment, the supplement comprises about 0.40%, or about 12 mg of EDTA.

Folic acid is a common form of vitamin B9. Vitamin B9 is essential for numerous bodily functions. As humans cannot synthesize vitamin B9 de novo, it must be supplied through the diet to meet daily requirements. Vitamin B9 assists in synthesizing DNA, repairing DNA, and acts as a cofactor in certain biological reactions. Children and adults both require folic acid to produce healthy red blood cells and prevent anemia.

In many embodiments of the present invention, a supplement comprises between about 0.1 mg to about 1.2 mg of folic acid, which may be a negligible amount up to about 0.04% of folic acid. In one particular embodiment, the supplement comprises about 0.4 mg of folic acid. Alternative embodiments of the present invention may utilize any form of Vitamin B9 as an alternative or supplement to folic acid.

Ginseng is still undergoing research in humans to determine its potential properties, but is believed to have therapeutic effects, such as for respiratory illnesses, quality of life, influenza or fatigue. In many embodiments of the present invention, a supplement comprises between about 0.33% to about 3.98%, or between about 10 mg to about 120 mg of ginseng. In one particular embodiment, the supplement comprises about 1.33%, or about 40 mg of ginseng. In one commercial embodiment, the ginseng may be provided in the form of commercially available as GinST-15, a proprietary formulation of ginseng.

Glucuronolactone is a chemical that is rapidly absorbed and metabolized into non-toxic metabolites such as xylulose. In addition, humans may be able to use glucuronolactone as a precursor for ascorbic acid synthesis. Glucuronolactone is known for its detoxification properties.

In many embodiments of the present invention, a supplement comprises between about 4.35% to about 52.25%, or between about 131.25 mg to about 1575 mg of glucuronolactone. In one particular embodiment, the supplement comprises about 17.42%, or about 525 mg of glucuronolactone.

L-Phenylalanine (LPA) is an electrically neutral amino acid, one of the twenty common amino acids used to biochemically form proteins, coded for by DNA. LPA is commonly consumed for its reputed analgesic and antidepressant effects. In many embodiments of the present invention, a supplement comprises between about 3.11% to about 37.32%, or between about 93.75 mg to about 1125 mg of LPA. In one particular embodiment, the supplement comprises about 16.58%, or about 500 mg of LPA.

Malic acid is a common food additive used for its sour or tart tasting properties. In many embodiments of the present invention, a supplement comprises between about 3.32% to about 39.81%, or between about 100 mg to about 1200 mg of malic acid. In one particular embodiment, the supplement comprises about 13.27%, or about 400 mg of malic acid.

N-acetyl 1-tyrosine is a form of the amino acid tyrosine, and is used to treat depression, attention deficit disorder (ADD), attention deficit-hyperactivity disorder (ADHD), the inability to stay awake (narcolepsy), and improving alertness following sleep deprivation. It is also used to treat stress, premenstrual syndrome (PMS), Parkinson's disease, Alzheimer's disease, chronic fatigue syndrome (CFS), alcohol and cocaine withdrawal, heart disease and stroke, ED (erectile dysfunction), loss of interest in sex, schizophrenia, and as a suntan agent and appetite suppressant.

In many embodiments of the present invention, a supplement comprises between about 3.32% to about 49.76%, or between about 100 mg to about 1500 mg of n-acetyl 1-tyrosine. In one particular embodiment, the supplement comprises about 33.16%, or about 1000 mg of n-acetyl 1-tyrosine.

Niacin, vitamin B3, is required for cell respiration, helps in the release of energy and metabolism of carbohydrates, fats, and proteins, proper circulation and healthy skin, functioning of the nervous system, and normal secretion of bile and stomach fluids. It is used in the synthesis of sex hormones, treating schizophrenia and other mental illnesses, and a memory-enhancer. Niacin given in pharmaceutical dosage improves the blood cholesterol profile, and has been used to clear the body of organic poisons, such as certain insecticides.

In many embodiments of the present invention, a supplement comprises between about 0.29% to about 3.48%, or between about 8.75 mg to about 105 mg of niacin. In one particular embodiment, the supplement comprises about 1.16%, or about 35 mg of niacin.

Potassium sorbate is the potassium salt of sorbic acid, is used as an additive to inhibit molds and yeasts in many foods, such as cheese, wine, yogurt, dried meats, apple cider, soft drinks and fruit drinks, and baked goods to increase stability and shelf life. In many embodiments of the present invention, a supplement comprises between about 0.25% to about 2.99%, or between about 7.5 mg to about 90 mg of potassium sorbate. In one particular embodiment, the supplement comprises about 1.0%, or about 30 mg of potassium sorbate.

Pyridoxine, also known as vitamin B6, assists in the balancing of sodium and potassium as well as promoting red blood cell production. It is linked to cardiovascular health by decreasing the formation of homocysteine. Pyridoxine may help balance hormonal changes in women and aid the immune system. Most notably, a lack of pyridoxine may cause anemia, nerve damage, seizures, skin problems, and sores in the mouth.

In many embodiments of the present invention, a supplement comprises between about 0.02% to about 0.20%, or between about 0.5 mg to about 6 mg of pyridoxine. In one particular embodiment, the supplement comprises about 0.07%, or about 2 mg of pyridoxine.

Sucralose is an artificial sweetener that is approximately 600 times as sweet as sucrose (i.e., table sugar), twice as sweet as saccharin, and three times as sweet as aspartame. In many embodiments of the present invention, a supplement comprises between about 1.58% to about 18.91%, or between about 47.5 mg to about 570 mg of sucralose. In one particular embodiment, the supplement comprises about 6.30%, or about 190 mg of sucralose.

Sodium benzoate is a preservative and is most widely used in acidic foods such as salad dressings (e.g., vinegar), carbonated drinks (e.g., carbonic acid), jams and fruit juices (e.g., citric acid), pickles, and condiments. In many embodiments of the present invention, a supplement comprises between about 0.25% to about 2.99%, or between about 7.5 mg to about 90 mg of sodium benzoate. In one particular embodiment, the supplement comprises about 1.0%, or about 30 mg of sodium benzoate.

Sodium chloride is the name of table salt, and has a plurality of common uses included as a flavorant and a preservative. In many embodiments of the present invention, a supplement comprises between about 0.33% to about 3.98%, or between about 10 mg to about 120 mg of sodium chloride. In one particular embodiment, the supplement comprises about 1.33%, or about 40 mg of sodium chloride.

Taurine is an organic acid, and has many fundamental biological roles such as conjugation of bile acids, antioxidation, osmoregulation, membrane stabilization and modulation of calcium signaling. It is essential for cardiovascular function, and development and function of skeletal muscle, the retina and the central nervous system.

In many embodiments of the present invention, a supplement comprises between about 4.35% to about 52.25%, or between about 131.25 mg to about 1575 mg of taurine. In one particular embodiment, the supplement comprises about 17.42%, or about 525 mg of taurine.

In accordance with embodiments of the present invention, the inactive ingredients of the supplement may include sweeteners or flavorants, such as MagnaSweet 110, which is an ammonia salt of Glycyrrhizic Acid, and manufactured by Mafco Worldwide Corporation. In exemplary embodiments, the flavorant may be added in small amounts, for example, between 0.02 ml to about 0.5 ml of flavorant, and in one embodiment, about 0.06 ml.

In another embodiment, other inactive ingredients, such as water, may optionally be provided in an amount of between 0.001 ml to about 180 ml, and in one embodiment, about 60 ml.

The supplement can be made in a variety of forms, such as pharmaceutical compositions (e.g., tablet, powder, suspension, liquid, capsule, and gel), nutritional beverages, puddings, confections (e.g., candy), ice cream, frozen confections and/or novelties or non-baked, extruded food products such as bars.

In another embodiment, the ingredients of the supplement can be administered separately, just by incorporating certain components (e.g., bitter tasting ones) into a capsule or tablet and the remaining ingredients provided as a powder or nutritional bar. In one exemplary embodiment, the supplement is provided in a homogenous liquid form. In such an embodiment, the supplement may be packaged in a single dose bottle, e.g., a shot-sized bottle about 50 ml to about 75 ml in volume. The supplement can be formulated for single or multiple daily administration.

While the foregoing is directed to embodiments of the present invention, other and further embodiments of the invention may be devised without departing from the basic scope thereof. For example, although numerous embodiments having various features have been described herein, combinations of such various features in other combinations not discussed herein are contemplated within the scope of embodiments of the present invention.

What is claimed is:

1. A nutritional energy supplement having a set of active ingredients, the active ingredients comprising:
between about 40 mg to about 480 mg of caffeine;
between about 62.5 mg to about 900 mg of citicoline;
between about 0.000 mg to about 0.03 mg of cyanocobalamin;
between about 3 mg to about 36 mg ethylenediaminetetraacetic acid;
between about 0.1 mg to about 1.2 mg of folic acid;
between about 10 mg to about 120 mg of ginseng;
between about 131.25 mg to about 1575 mg of glucuronolactone;
between about 93.75 mg to about 1125 mg of L-Phenylalanine;
between about 100 mg to about 1200 mg of malic acid;
between about 100 mg to about 1500 mg of n-acetyl 1-tyrosine;
between about 8.75 mg to about 105 mg of niacin;
between about 7.5 mg to about 90 mg of potassium sorbate; and
between about 0.5 mg to about 6 mg of pyridoxine.

2. The nutritional energy supplement of claim 1, further having a set of inactive ingredients, the inactive ingredients comprising:
between about 47.5 mg to about 570 mg of sucralose;
between about 7.5 mg to about 90 mg of sodium benzoate;
between about 10 mg to about 120 mg of sodium chloride;
between about 131.25 mg to about 1575 mg of taurine;
between about 0.02 ml to about 0.5 ml of flavorant; and
between about 0.001 ml to about 180 ml of water.

3. The nutritional energy supplement of claim 1, wherein the supplement is prepared as one of a tablet, a powder, a suspension, a liquid, a capsule, a gel, a beverage, a pudding, a confection, an ice cream, a frozen confection or a non-baked, extruded food bar.

4. The nutritional energy supplement of claim 3, wherein the supplement is prepared in liquid form, and packaged in at least about a 50 ml to about a 75 ml bottle.

5. The nutritional energy supplement of claim 1, wherein the active ingredients comprise:
about 160 mg of caffeine;
about 500 mg of citicoline;
about 0.01 mg of cyanocobalamin;
about 12 mg ethylenediaminetetraacetic acid;
about 0.4 mg of folic acid;
about 50 mg of ginseng;
about 525 mg of glucuronolactone;
about 500 mg of L-Phenylalanine;
about 400 mg of malic acid;
about 1000 mg of n-acetyl 1-tyrosine;
about 35 mg of niacin;
about 30 mg of potassium sorbate; and
about 2 mg of pyridoxine.

6. The nutritional energy supplement of claim 5, further having a set of inactive ingredients, the inactive ingredients comprising:
about 190 mg of sucralose;
about 30 mg of sodium benzoate;
about 40 mg of sodium chloride;
about 525 mg of taurine;
about 0.06 ml of flavorant; and
about 60 ml of water.

7. A nutritional energy supplement comprising:
between about 40 mg to about 480 mg of caffeine;
between about 62.5 mg to about 900 mg of citicoline;
between about 0.000 mg to about 0.03 mg of cyanocobalamin;
between about 3 mg to about 36 mg ethylenediaminetetraacetic acid;
between about 0.1 mg to about 1.2 mg of folic acid;
between about 10 mg to about 120 mg of ginseng;
between about 131.25 mg to about 1575 mg of glucuronolactone;
between about 93.75 mg to about 1125 mg of L-Phenylalanine;
between about 100 mg to about 1200 mg of malic acid;
between about 100 mg to about 1500 mg of n-acetyl 1-tyrosine;
between about 8.75 mg to about 105 mg of niacin;
between about 7.5 mg to about 90 mg of potassium sorbate;
between about 0.5 mg to about 6 mg of pyridoxine;
between about 47.5 mg to about 570 mg of sucralose;
between about 7.5 mg to about 90 mg of sodium benzoate;
between about 10 mg to about 120 mg of sodium chloride;
between about 131.25 mg to about 1575 mg of taurine;
between about 0.02 ml to about 0.5 ml of flavorant; and
between about 0.001 ml to about 180 ml of water.

8. The nutritional energy supplement of claim 7, wherein the supplement comprises:
about 160 mg of caffeine;
about 500 mg of citicoline;
about 0.01 mg of cyanocobalamin;
about 12 mg ethylenediaminetetraacetic acid;
about 0.4 mg of folic acid;
about 50 mg of ginseng;
about 525 mg of glucuronolactone;
about 500 mg of L-Phenylalanine;
about 400 mg of malic acid;
about 1000 mg of n-acetyl 1-tyrosine;
about 35 mg of niacin;
about 30 mg of potassium sorbate;
about 2 mg of pyridoxine;
about 190 mg of sucralose;
about 30 mg of sodium benzoate;
about 40 mg of sodium chloride;
about 525 mg of taurine;
about 0.06 ml of flavorant; and
about 60 ml of water.

9. A nutritional energy supplement consisting of:
between about 40 mg to about 480 mg of caffeine;
between about 62.5 mg to about 900 mg of citicoline;
between about 0.000 mg to about 0.03 mg of cyanocobalamin;
between about 3 mg to about 36 mg ethylenediaminetetraacetic acid;
between about 0.1 mg to about 1.2 mg of folic acid;
between about 10 mg to about 120 mg of ginseng;
between about 131.25 mg to about 1575 mg of glucuronolactone;
between about 93.75 mg to about 1125 mg of L-Phenylalanine;
between about 100 mg to about 1200 mg of malic acid;
between about 100 mg to about 1500 mg of n-acetyl 1-tyrosine;
between about 8.75 mg to about 105 mg of niacin;
between about 7.5 mg to about 90 mg of potassium sorbate;
between about 0.5 mg to about 6 mg of pyridoxine;
between about 47.5 mg to about 570 mg of sucralose;
between about 7.5 mg to about 90 mg of sodium benzoate;
between about 10 mg to about 120 mg of sodium chloride;
between about 131.25 mg to about 1575 mg of taurine;
between about 0.02 ml to about 0.5 ml of flavorant; and
between about 0.001 ml to about 180 ml of water.

10. The nutritional energy supplement of claim 9, wherein the supplement consists of:
about 160 mg of caffeine;
about 500 mg of citicoline;
about 0.01 mg of cyanocobalamin;
about 12 mg ethylenediaminetetraacetic acid;
about 0.4 mg of folic acid;
about 50 mg of ginseng;
about 525 mg of glucuronolactone;
about 500 mg of L-Phenylalanine;
about 400 mg of malic acid;
about 1000 mg of n-acetyl 1-tyrosine;
about 35 mg of niacin;
about 30 mg of potassium sorbate;
about 2 mg of pyridoxine;
about 190 mg of sucralose;
about 30 mg of sodium benzoate;
about 40 mg of sodium chloride;
about 525 mg of taurine;
about 0.06 ml of flavorant; and
about 60 ml of water.
